(19) United States
(12) Patent Application Publication
(10) Pub. No.: US 2026/0217630 A1
(43) Pub. Date: Jul. 30, 2026

(54) SEPARATION METHOD FOR POLYCYCLIC AROMATIC HYDROCARBONS

(71) Applicant: TAIYUAN UNIVERSITY OF TECHNOLOGY, Taiyuan (CN)

(72) Inventors: Wenying LI, Taiyuan (CN); Pengzhi BEI, Taiyuan (CN); Jie FENG, Taiyuan (CN); Xiaohong LI, Taiyuan (CN)

(73) Assignee: TAIYUAN UNIVERSITY OF TECHNOLOGY, Taiyuan (CN)

(21) Appl. No.: 19/142,656

(22) PCT Filed: Aug. 22, 2024
(86) PCT No.: PCT/CN2024/113862

§ 371 Date: Jun. 24, 2025

(30) Foreign Application Priority Data
Oct. 19, 2023 (CN) 202311360110.4

(51) Int. Cl. C07C 7/00; C07C 7/04; C07C 7/10; C07C 15/24; C07C 15/27; C07C 15/38

(52) U.S. Cl. CPC C07C 7/005; C07C 7/04; C07C 7/10; C07C 15/24; C07C 15/27; C07C 15/38

(57) ABSTRACT

A separation method for polycyclic aromatic hydrocarbons is provided. A mixed solvent and a to-be-separated mixture are first mixed and then filtered to obtain a filter cake and a filtrate, and then a back extraction agent and the filtrate are mixed and separated, where the mixed solvent is a naphthalene-based mixed solvent or an imidazole-based mixed solvent, and the to-be-separated mixture is a mixture containing bicyclic aromatic hydrocarbons and tricyclic aromatic hydrocarbons or a mixture containing tricyclic aromatic hydrocarbons and tetracyclic aromatic hydrocarbons. A mixture containing bicyclic aromatic hydrocarbons and tricyclic aromatic hydrocarbons is separated by using a naphthalene-based mixed solvent, and a mixture containing tricyclic aromatic hydrocarbons and tetracyclic aromatic hydrocarbons is separated by using an imidazole-based mixed solvent, and the naphthalene-based mixed solvent or the imidazole-based mixed solvent can be recycled. In addition, the separation method has simple steps, low energy consumption and a simple process flow.

### CROSS REFERENCE TO THE RELATED APPLICATIONS

This application is the national phase entry of International Application No. PCT/CN2024/113862, filed on Aug. 22, 2024, which is based upon and claims priority to Chinese Patent Application No. 202311360110.4, filed on Oct. 19, 2023, the entire contents of which are incorporated herein by reference.

### TECHNICAL FIELD

The present invention relates to the technical field of separation of polycyclic aromatic hydrocarbons, and particularly to a separation method for polycyclic aromatic hydrocarbons.

### BACKGROUND

Polycyclic aromatic hydrocarbons are the most basic raw materials in the chemical industry, which have high application value and may be used in many research fields. Tricyclic aromatic hydrocarbons and tetracyclic aromatic hydrocarbons have good fluorescence effects and may be used as raw materials for preparing photoelectric materials. Meanwhile, the tricyclic aromatic hydrocarbons and the tetracyclic aromatic hydrocarbons may also be used as additives for high-energy density fuels after hydrogenation to improve fuel performance. Coal tar is rich in polycyclic aromatic hydrocarbons, among which dicyclic aromatic hydrocarbons, tricyclic aromatic hydrocarbons and tetracyclic aromatic hydrocarbons are more abundant.

At present, the main separation methods include distillation, supercritical extraction, zone melting, column chromatography separation and extraction, among which distillation is the most widely used, but has disadvantages such as high energy consumption and long process flow. The extraction is simple to operate and has low requirements on equipment. However, since obvious recognition sites for dicyclic aromatic hydrocarbons, tricyclic aromatic hydrocarbons and tetracyclic aromatic hydrocarbons are difficult to find, a proper solvent is difficult to find for separating the polycyclic aromatic hydrocarbons at present. Therefore, it is of great significance to provide a separation method for polycyclic aromatic hydrocarbons to achieve the separation of a mixture of dicyclic aromatic hydrocarbons and tricyclic aromatic hydrocarbons and the separation of a mixture of tricyclic aromatic hydrocarbons and tetracyclic aromatic hydrocarbons.

### SUMMARY

An objective of the present invention is to provide a separation method for polycyclic aromatic hydrocarbons to solve the problems of complicated separation steps and high energy consumption in the prior art for separating a mixture containing bicyclic aromatic hydrocarbons and tricyclic aromatic hydrocarbons or a mixture containing tricyclic aromatic hydrocarbons and tetracyclic aromatic hydrocarbons.

To achieve the above objective, the present invention provides the following technical solutions.

The present invention provides a separation method for polycyclic aromatic hydrocarbons, which includes the following steps:

(1) mixing a mixed solvent and a to-be-separated mixture, and filtering to obtain a filter cake and a filtrate;

(2) mixing a back extraction agent and the filtrate and then performing separation; wherein the mixed solvent is a naphthalene-based mixed solvent or an imidazole-based mixed solvent; and

the to-be-separated mixture is a mixture containing bicyclic aromatic hydrocarbons and tricyclic aromatic hydrocarbons or a mixture containing tricyclic aromatic hydrocarbons and tetracyclic aromatic hydrocarbons.

Preferably, in the step (1), a mass ratio of the mixed solvent to the to-be-separated mixture is (1-8):1.

Preferably, the mixture containing bicyclic aromatic hydrocarbons and tricyclic aromatic hydrocarbons is separated by using a naphthalene-based mixed solvent, wherein the naphthalene-based mixed solvent is formed by mixing an alcohol compound and a naphthyl compound.

Preferably, a concentration of the naphthyl compound is 2-20 mol %.

Preferably, the naphthyl compound includes one or more of naphthylacetonitrile, naphthylacetic acid, naphthylacetamide, naphthylmethanol, naphthylethanol, naphthylamine and naphthol.

Preferably, the mixture containing the tricyclic aromatic hydrocarbons and the tetracyclic aromatic hydrocarbons is separated by using an imidazole-based mixed solvent, wherein the imidazole-based mixed solvent is formed by mixing an alcohol compound and an imidazole compound.

Preferably, the alcohol compound includes one or more of ethylene glycol, propylene glycol, pentanediol, heptanediol and octanediol.

Preferably, the imidazole compound includes one or more of methylimidazole, ethylimidazole, propylimidazole and butylimidazole.

Preferably, a concentration of the imidazole compound is 10-80 mol %.

The beneficial effects of the present invention are as follows.

(1) According to the present invention, a mixture containing bicyclic aromatic hydrocarbons and tricyclic aromatic hydrocarbons is separated by using a naphthalene-based mixed solvent, and a mixture containing tricyclic aromatic hydrocarbons and tetracyclic aromatic hydrocarbons is separated by using an imidazole-based mixed solvent, and the naphthalene-based mixed solvent or the imidazole-based mixed solvent can be recycled.

(2) The separation method provided by the present invention has the advantages of simple steps, low energy consumption, simple process flow and low solvent toxicity, and the used naphthalene-based mixed solvent and imidazole-based mixed solvent may be efficiently utilized by recycling and regeneration through a back extraction method.

### DETAILED DESCRIPTION OF THE EMBODIMENTS

The present invention provides a separation method for polycyclic aromatic hydrocarbons, which includes the following steps:

(1) mixing a mixed solvent and a to-be-separated mixture, and filtering to obtain a filter cake and a filtrate;

(2) mixing a back extraction agent and the filtrate and then performing separation; wherein the mixed solvent is a naphthalene-based mixed solvent or an imidazole-based mixed solvent; and

the to-be-separated mixture is a mixture containing bicyclic aromatic hydrocarbons and tricyclic aromatic hydrocarbons or a mixture containing tricyclic aromatic hydrocarbons and tetracyclic aromatic hydrocarbons.

In the present invention, the mixture containing bicyclic aromatic hydrocarbons and tricyclic aromatic hydrocarbons is a model mixture containing bicyclic aromatic hydrocarbons and tricyclic aromatic hydrocarbons or a fraction of coal tar at 220-320° C., preferably a model mixture containing bicyclic aromatic hydrocarbons and tricyclic aromatic hydrocarbons.

In the present invention, the model mixture containing bicyclic aromatic hydrocarbons and tricyclic aromatic hydrocarbons is a mixture of two or more of naphthalene, methylnaphthalene, anthracene, phenanthrene and fluorene, preferably a mixture of naphthalene and phenanthrene, a mixture of naphthalene and anthracene, or a mixture of naphthalene, phenanthrene and fluorene. In the present invention, the mixture containing tricyclic aromatic hydrocarbons and tetracyclic aromatic hydrocarbons is a model mixture containing tricyclic aromatic hydrocarbons and tetracyclic aromatic hydrocarbons or a fraction of coal tar at 300-400° C., preferably a model mixture containing tricyclic aromatic hydrocarbons and tetracyclic aromatic hydrocarbons.

In the present invention, the model mixture containing tricyclic aromatic hydrocarbons and tetracyclic aromatic hydrocarbons is a mixture of two or more of anthracene, phenanthrene, fluorene, pyrene and chrysene, preferably a mixture of phenanthrene and pyrene, or a mixture of phenanthrene, pyrene and chrysene, or a mixture of phenanthrene, anthracene, pyrene and chrysene, or a mixture of phenanthrene, anthracene, fluorene, pyrene and chrysene.

In the present invention, in the step (1), a mass ratio of the mixed solvent to the to-be-separated mixture is (1-8):1, preferably 1:1, 2:1, 3:1, 5:1, 6:1, and preferably 3:1 or 5:1.

In the present invention, in the step (1), the mixed solvent and the to-be-separated mixture are mixed and then stirred, wherein the stirring temperature is controlled at 20-90° C., preferably 30-80° C., preferably 30° C., 40° C., 50° C., 70° C.

In the present invention, in the step (2), the back-extraction agent and the filtrate are mixed and stirred, wherein the stirring temperature is controlled at 20-40° C., preferably 25-35° C., and more preferably 30° C.

In the present invention, the mixture containing bicyclic aromatic hydrocarbons and tricyclic aromatic hydrocarbons is separated by using a naphthalene-based mixed solvent, wherein the naphthalene-based mixed solvent is formed by mixing an alcohol compound and a naphthyl compound.

In the present invention, the alcohol compound in the naphthalene-based mixed solvent includes one or more of ethylene glycol, propylene glycol, pentanediol, heptanediol and octanediol, preferably one or more of ethylene glycol, propylene glycol and pentanediol, and more preferably ethylene glycol.

In the present invention, the concentration of the naphthyl compound is 2-20 mol %, preferably 5-18 mol %, and more preferably 10-15 mol %.

In the present invention, the naphthyl compound includes one or more of naphthylacetonitrile, naphthylacetic acid, naphthylacetamide, naphthylmethanol, naphthylethanol, naphthylamine and naphthol, preferably one or more of naphthylacetonitrile, naphthylacetic acid, naphthylmethanol and naphthol, and more preferably naphthylmethanol.

In the present invention, the back extraction agent used for separating the mixture containing bicyclic aromatic hydrocarbons and tricyclic aromatic hydrocarbons is one or more of n-hexane, n-heptane, n-octane, n-nonane and n-decane, preferably one or more of n-hexane, n-heptane, n-octane and n-nonane, and more preferably n-hexane and/or n-heptane.

In the present invention, in the separation of the mixture containing bicyclic aromatic hydrocarbons and tricyclic aromatic hydrocarbons, the mass ratio of the back extraction agent to the filtrate is (2-20):1, preferably (5-15):1, and more preferably (8-12):1.

In the present invention, the mixture containing bicyclic aromatic hydrocarbons and tricyclic aromatic hydrocarbons is separated to obtain a mixture of a back extraction agent and bicyclic aromatic hydrocarbons, which are subjected to distillation under reduced pressure to obtain the back extraction agent and bicyclic aromatic hydrocarbons, wherein the temperature of the distillation under reduced pressure is 30-70° C., preferably 40-60° C., and more preferably 50 ° C., and the time is 30-90 min, preferably 40-80 min, and more preferably 50-70 min.

In the present invention, the mixture containing the tricyclic aromatic hydrocarbons and the tetracyclic aromatic hydrocarbons is separated by using an imidazole-based mixed solvent, wherein the imidazole-based mixed solvent is formed by mixing an alcohol compound and an imidazole compound.

In the present invention, the alcohol compound includes one or more of ethylene glycol, propylene glycol, pentanediol, heptanediol and octanediol, preferably one or more of ethylene glycol, propylene glycol and pentanediol, and more preferably ethylene glycol.

In the present invention, the imidazole compound includes one or more of methylimidazole, ethylimidazole, propylimidazole and butylimidazole, preferably one or more of ethylimidazole, propylimidazole and butylimidazole, and more preferably ethylimidazole and/or butylimidazole. In the present invention, the concentration of the imidazole compound is 10-80 mol %, preferably 15-75 mol %, and more preferably 20-60 mol %.

In the present invention, for the separation of the mixture containing tricyclic aromatic hydrocarbons and tetracyclic aromatic hydrocarbons, the back extraction agent is preferably ethylene glycol, wherein the mixing of ethylene glycol and the filtrate is preferably performed by slowly adding ethylene glycol to the filtrate.

The technical solutions provided in the present invention will be described in detail below with reference to the examples, which, however, should not be construed as limiting the scope of the present invention.

### Example 1

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthylmethanol and ethylene glycol, and a concentration of naphthylmethanol was 10 mol %) and a mixture of naphthalene and phenanthrene were mixed in a mass ratio of 1:1 and stirred at 30° C. Finally, the mixture was filtered to obtain a filter cake (tricyclic aromatic hydrocarbons) and a filtrate.

The n-hexane and the filtrate were mixed in a mass ratio of 10:1 and stirred at 30° C. for 50 min. Finally, the naphthalene-based mixed solvent and the mixture of n-hexane and bicyclic aromatic hydrocarbons were separated by washing and filtration.

The mixture of n-hexane and bicyclic aromatic hydrocarbons was distilled under reduced pressure at 50° C. for 50 min to obtain the n-hexane and the bicyclic aromatic hydrocarbons. The purity of the obtained bicyclic aromatic hydrocarbon compound was 89.1% and the yield was 60%. The purity of the tricyclic aromatic hydrocarbon compound was 87.2% and the yield was 60%.

### Example 2

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthylmethanol and ethylene glycol, and a concentration of naphthylmethanol was 10 mol %) and a mixture of naphthalene and phenanthrene were mixed in a mass ratio of 2:1 and stirred at 30° C. Finally, the mixture was filtered to obtain a filter cake (tricyclic aromatic hydrocarbons) and a filtrate.

The n-heptane and the filtrate were mixed in a mass ratio of 15:1 and stirred at 30° C. for 50 min. Finally, the naphthalene-based mixed solvent and the mixture of n-heptane and bicyclic aromatic hydrocarbons were separated by washing and filtration.

The mixture of n-heptane and bicyclic aromatic hydrocarbons was distilled under reduced pressure at 60° C. for 40 min to obtain the n-heptane and the bicyclic aromatic hydrocarbons. The purity of the obtained bicyclic aromatic hydrocarbon compound was 90.5% and the yield was 62%. The purity of the tricyclic aromatic hydrocarbon compound was 89.5% and the yield was 61%.

### Example 3

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthylmethanol and ethylene glycol, and a concentration of naphthylmethanol was 10 mol %) and a mixture of naphthalene and phenanthrene were mixed in a mass ratio of 3:1 and stirred at 50° C. Finally, the mixture was filtered to obtain a filter cake (tricyclic aromatic hydrocarbons) and a filtrate.

The n-octane and the filtrate were mixed in a mass ratio of 5:1 and stirred at 50° C. for 50 min. Finally, the naphthalene-based mixed solvent and the mixture of n-octane and bicyclic aromatic hydrocarbons were separated by washing and filtration.

The mixture of n-octane and bicyclic aromatic hydrocarbons was distilled under reduced pressure at 30° C. for 90 min to obtain the n-octane and the bicyclic aromatic hydrocarbons. The purity of the obtained bicyclic aromatic hydrocarbon compound was 94.5% and the yield was 67%. The purity of the tricyclic aromatic hydrocarbon compound was 94.9% and the yield was 68%.

### Example 4

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthylmethanol and ethylene glycol, and a concentration of naphthylmethanol was 15 mol %) and a mixture of naphthalene and phenanthrene were mixed in a mass ratio of 5:1 and stirred at 30° C. Finally, the mixture was filtered to obtain a filter cake (tricyclic aromatic hydrocarbons) and a filtrate.

The n-octane and the filtrate were mixed in a mass ratio of 20:1 and stirred at 20° C. for 90 min. Finally, the naphthalene-based mixed solvent and the mixture of n-octane and bicyclic aromatic hydrocarbons were separated by washing and filtration.

The mixture of n-octane and bicyclic aromatic hydrocarbons was distilled under reduced pressure at 30° C. for 90 min to obtain the n-octane and the bicyclic aromatic hydrocarbons. The purity of the obtained bicyclic aromatic hydrocarbon compound was 92.5% and the yield was 65%. The purity of the tricyclic aromatic hydrocarbon compound was 93.1% and the yield was 66%.

### Example 5

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthylacetonitrile and ethylene glycol, and a concentration of naphthylacetonitrile was 8 mol %) and a mixture of naphthalene and phenanthrene were mixed in a mass ratio of 2:1 and stirred at 30° C. Finally, the mixture was filtered to obtain a filter cake (tricyclic aromatic hydrocarbons) and a filtrate.

The n-octane and the filtrate were mixed in a mass ratio of 20:1 and stirred at 20° C. for 90 min. Finally, the naphthalene-based mixed solvent and the mixture of n-octane and bicyclic aromatic hydrocarbons were separated by washing and filtration.

The mixture of n-octane and bicyclic aromatic hydrocarbons was distilled under reduced pressure at 30° C. for 90 min to obtain the n-octane and the bicyclic aromatic hydrocarbons. The purity of the obtained bicyclic aromatic hydrocarbon compound was 90.5% and the yield was 62%. The purity of the tricyclic aromatic hydrocarbon compound was 91.6% and the yield was 62%.

### Example 6

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthylacetic acid and ethylene glycol, and a concentration of naphthylacetic acid was 12 mol %) and a mixture of naphthalene and phenanthrene were mixed in a mass ratio of 3:1 and stirred at 30° C. Finally, the mixture was filtered to obtain a filter cake (tricyclic aromatic hydrocarbons) and a filtrate.

The n-octane and the filtrate were mixed in a mass ratio of 20:1 and stirred at 20° C. for 90 min. Finally, the naphthalene-based mixed solvent and the mixture of n-octane and bicyclic aromatic hydrocarbons were separated by washing and filtration.

The mixture of n-octane and bicyclic aromatic hydrocarbons was distilled under reduced pressure at 50° C. for 60 min to obtain the n-octane and the bicyclic aromatic hydrocarbons. The purity of the obtained bicyclic aromatic hydrocarbon compound was 91.8% and the yield was 61%. The purity of the tricyclic aromatic hydrocarbon compound was 92.8% and the yield was 64%.

### Example 7

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthylacetamide and ethylene glycol, and a concentration of naphthylacetamide was 18 mol %) and a mixture of naphthalene and phenanthrene were mixed in a mass ratio of 6:1 and stirred at 70° C. Finally, the mixture was filtered to obtain a filter cake (tricyclic aromatic hydrocarbons) and a filtrate.

The n-octane and the filtrate were mixed in a mass ratio of 20:1 and stirred at 20° C. for 90 min. Finally, the naphthalene-based mixed solvent and the mixture of n-octane and bicyclic aromatic hydrocarbons were separated by washing and filtration.

The mixture of n-octane and bicyclic aromatic hydrocarbons was distilled under reduced pressure at 40° C. for 30 min to obtain the n-octane and the bicyclic aromatic hydrocarbons. The purity of the obtained bicyclic aromatic hydrocarbon compound was 86.8% and the yield was 55%. The purity of the tricyclic aromatic hydrocarbon compound was 89.1% and the yield was 61%.

### Example 8

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthylethanol and ethylene glycol, and a concentration of naphthylethanol was 18 mol %) and a mixture of naphthalene and phenanthrene were mixed in a mass ratio of 8:1 and stirred at 40° C. Finally, the mixture was filtered to obtain a filter cake (tricyclic aromatic hydrocarbons) and a filtrate.

The n-octane and the filtrate were mixed in a mass ratio of 20:1 and stirred at 20° C. for 90 min. Finally, the naphthalene-based mixed solvent and the mixture of n-octane and bicyclic aromatic hydrocarbons were separated by washing and filtration.

The mixture of n-octane and bicyclic aromatic hydrocarbons was distilled under reduced pressure at 70° C. for 30 min to obtain the n-octane and the bicyclic aromatic hydrocarbons. The purity of the obtained bicyclic aromatic hydrocarbon compound was 89.6% and the yield was 60%. The purity of the tricyclic aromatic hydrocarbon compound was 90.5% and the yield was 63%.

### Example 9

2 g of naphthalene and 1 g of phenanthrene were mixed in dichloromethane, stirred and heated at room temperature. The dichloromethane was distilled to obtain a mixture of naphthalene and phenanthrene. A naphthalene-based mixed solvent (wherein the a naphthalene-based mixed solvent was a mixture of naphthol and ethylene glycol, and a concentration of naphthol was 18 mol %) and a mixture of naphthalene and phenanthrene were mixed in a mass ratio of 3:1 and stirred at 50° C. Finally, the mixture was filtered to obtain a filter cake (tricyclic aromatic hydrocarbons) and a filtrate.

The n-octane and the filtrate were mixed in a mass ratio of 20:1 and stirred at 20° C. for 90 min. Finally, the naphthalene-based mixed solvent and the mixture of n-octane and bicyclic aromatic hydrocarbons were separated by washing and filtration.

The mixture of n-octane and bicyclic aromatic hydrocarbons was distilled under reduced pressure at 30° C. for 90 min to obtain the n-octane and the bicyclic aromatic hydrocarbons. The purity of the obtained bicyclic aromatic hydrocarbon compound was 91.3% and the yield was 61%. The purity of the tricyclic aromatic hydrocarbon compound was 93.3% and the yield was 63%.

### Example 10

2 g of naphthalene and 1 g of anthracene were mixed in dichloromethane, stirred and heated at room temperature. The dichloromethane was distilled to obtain a mixture of naphthalene and anthracene. A naphthalene-based mixed solvent (wherein the a naphthalene-based mixed solvent was a mixture of naphthylmethanol and ethylene glycol, and a concentration of naphthylmethanol was 5 mol %) and a mixture of naphthalene and anthracene were mixed in a mass ratio of 3:1 and stirred at 40° C. Finally, the mixture was filtered to obtain a filter cake (tricyclic aromatic hydrocarbons) and a filtrate.

The n-octane and the filtrate were mixed in a mass ratio of 20:1 and stirred at 20° C. for 90 min. Finally, the naphthalene-based mixed solvent and the mixture of n-octane and bicyclic aromatic hydrocarbons were separated by washing and filtration.

The mixture of n-octane and bicyclic aromatic hydrocarbons was distilled under reduced pressure at 30° C. for 90 min to obtain the n-octane and the bicyclic aromatic hydrocarbons. The purity of the obtained bicyclic aromatic hydrocarbon compound was 96.5% and the yield was 67%. The purity of the tricyclic aromatic hydrocarbon compound was 96.8% and the yield was 66%.

### Example 11

2 g of naphthalene, 0.5 g of phenanthrene, 0.3 g of anthracene and 0.2 g of fluorene were mixed in dichloromethane, stirred and heated at room temperature. The dichloromethane was distilled to obtain a mixture of naphthalene, phenanthrene, anthracene and fluorene. A naphthalene-based mixed solvent (wherein the naphthalene-based mixed solvent was a mixture of naphthylmethanol and ethylene glycol, and the concentration of naphthylmethanol was 10 mol %) and a mixture of naphthalene, phenanthrene, anthracene and fluorene were mixed in a mass ratio of 3:1 and stirred at 50° C. Finally, the mixture was filtered to obtain a filter cake (tricyclic aromatic hydrocarbons) and a filtrate.

The n-hexane and the filtrate were mixed in a mass ratio of 20:1 and stirred at 20° C. for 90 min. Finally, the naphthalene-based mixed solvent and the mixture of n-hexane and bicyclic aromatic hydrocarbons were separated by washing and filtration.

The mixture of n-hexane and bicyclic aromatic hydrocarbons was distilled under reduced pressure at 30° C. for 90 min to obtain the n-hexane and the bicyclic aromatic hydrocarbons. The purity of the obtained bicyclic aromatic hydrocarbon compound was 95.4% and the yield was 66%. The purity of the tricyclic aromatic hydrocarbon compound was 96.5% and the yield was 67%.

### Example 12

2 g of phenanthrene and 1 g of pyrene were mixed in dichloromethane, stirred and heated at room temperature. The dichloromethane was distilled to remove the dichloromethane to obtain a mixture of phenanthrene and pyrene. An imidazole-based mixed solvent (wherein the imidazole-based mixed solvent was a mixture of ethylimidazole and ethylene glycol, and the concentration of ethylimidazole was 30 mol %) and a mixture of phenanthrene and pyrene were mixed in a mass ratio of 1:1, and then stirred at 30° C. Finally, the mixture was filtered to obtain a filter cake (tricyclic aromatic hydrocarbons) and a filtrate.

Ethylene glycol was slowly added to the filtrate, and the temperature was controlled at 30 ° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 12 was 84.5%, and the yield was 58%. The purity of the tetracyclic aromatic hydrocarbons obtained was 83.5%, and the yield was 52%.

### Example 13

2 g of phenanthrene and 1 g of pyrene were mixed in dichloromethane, stirred and heated at room temperature. The dichloromethane was distilled to remove the dichloromethane to obtain a mixture of phenanthrene and pyrene. An imidazole-based mixed solvent (wherein the imidazole-based mixed solvent was a mixture of ethylimidazole and propylene glycol, and the concentration of ethylimidazole was 40 mol %) and a mixture of phenanthrene and pyrene were mixed in a mass ratio of 2:1, and then stirred at 40° C. Finally, the mixture was filtered to obtain a filter cake (tricyclic aromatic hydrocarbons) and a filtrate.

Ethylene glycol was slowly added to the filtrate, and the temperature was controlled at 40° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 13 was 88.4%, and the yield was 60%. The purity of the tetracyclic aromatic hydrocarbons obtained was 86.5%, and the yield was 58%.

### Example 14

2 g of phenanthrene and 1 g of pyrene were mixed in dichloromethane, stirred and heated at room temperature. The dichloromethane was distilled to remove the dichloromethane to obtain a mixture of phenanthrene and pyrene. An imidazole-based mixed solvent (wherein the imidazole-based mixed solvent was a mixture of ethylimidazole and ethylene glycol, and the concentration of ethylimidazole was 40 mol %) and a mixture of phenanthrene and pyrene were mixed in a mass ratio of 3:1, and then stirred at 40° C. Finally, the mixture was filtered to obtain a filter cake (tricyclic aromatic hydrocarbons) and a filtrate.

Ethylene glycol was slowly added to the filtrate, and the temperature was controlled at 20° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 14 was 86.4%, and the yield was 58%. The purity of the tetracyclic aromatic hydrocarbons obtained was 83.5%, and the yield was 52%.

### Example 15

2 g of phenanthrene and 1 g of pyrene were mixed in dichloromethane, stirred and heated at room temperature. The dichloromethane was distilled to remove the dichloromethane to obtain a mixture of phenanthrene and pyrene. An imidazole-based mixed solvent (wherein the imidazole-based mixed solvent was a mixture of ethylimidazole and ethylene glycol, and the concentration of ethylimidazole was 40 mol %) and a mixture of phenanthrene and pyrene were mixed in a mass ratio of 5:1, and then stirred at 30° C. Finally, the mixture was filtered to obtain a filter cake (tricyclic aromatic hydrocarbons) and a filtrate.

Ethylene glycol was slowly added to the filtrate, and the temperature was controlled at 30° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 15 was 81.9%, and the yield was 52%. The purity of the tetracyclic aromatic hydrocarbons obtained was 80.4%, and the yield was 52%.

### Example 16

2 g of phenanthrene and 1 g of pyrene were mixed in dichloromethane, stirred and heated at room temperature. The dichloromethane was distilled to remove the dichloromethane to obtain a mixture of phenanthrene and pyrene. An imidazole-based mixed solvent (wherein the imidazole-based mixed solvent was a mixture of methylimidazole and pentanediol, and the concentration of methylimidazole was 50 mol %) and a mixture of phenanthrene and pyrene were mixed in a mass ratio of 2:1, and then stirred at 40° C. Finally, the mixture was filtered to obtain a filter cake (tricyclic aromatic hydrocarbons) and a filtrate.

Ethylene glycol was slowly added to the filtrate, and the temperature was controlled at 30 ° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 16 was 84.2%, and the yield was 56%. The purity of the tetracyclic aromatic hydrocarbons obtained was 83.9%, and the yield was 55%.

### Example 17

2 g of phenanthrene and 1 g of pyrene were mixed in dichloromethane, stirred and heated at room temperature. The dichloromethane was distilled to remove the dichloromethane to obtain a mixture of phenanthrene and pyrene. An imidazole-based mixed solvent (wherein the imidazole-based mixed solvent was a mixture of propylimidazole and ethylene glycol, and the concentration of propylimidazole was 20 mol %) and a mixture of phenanthrene and pyrene were mixed in a mass ratio of 2:1, and then stirred at 40° C. Finally, the mixture was filtered to obtain a filter cake (tricyclic aromatic hydrocarbons) and a filtrate.

Ethylene glycol was slowly added to the filtrate, and the temperature was controlled at 30 ° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 17 was 83.9%, and the yield was 57%. The purity of the tetracyclic aromatic hydrocarbons obtained was 84.9%, and the yield was 59%.

### Example 18

2 g of phenanthrene and 1 g of pyrene were mixed in dichloromethane, stirred and heated at room temperature. The dichloromethane was distilled to remove the dichloromethane to obtain a mixture of phenanthrene and pyrene. An imidazole-based mixed solvent (wherein the imidazole-based mixed solvent was a mixture of butylimidazole and octanediol, and the concentration of butylimidazole was 60 mol %) and a mixture of phenanthrene and pyrene were mixed in a mass ratio of 8:1, and then stirred at 40° C. Finally, the mixture was filtered to obtain a filter cake (tricyclic aromatic hydrocarbons) and a filtrate.

Ethylene glycol was slowly added to the filtrate, and the temperature was controlled at 30 ° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 18 was 83.2%, and the yield was 58%. The purity of the tetracyclic aromatic hydrocarbons obtained was 82.7%, and the yield was 56%.

### Example 19

2 g of phenanthrene, 0.5 g of pyrene and 0.5 g of chrysene were mixed in dichloromethane, stirred and heated at room temperature. The dichloromethane was distilled to remove the dichloromethane to obtain a mixture of phenanthrene, pyrene and chrysene. An imidazole-based mixed solvent (wherein the imidazole-based mixed solvent was a mixture of ethylimidazole and ethylene glycol, and the concentration of ethylimidazole was 40 mol %) and a mixture of phenanthrene, pyrene and chrysene were mixed in a mass ratio of 2:1, and then stirred at 50° C. Finally, the mixture was filtered to obtain a filter cake (tricyclic aromatic hydrocarbons) and a filtrate.

Ethylene glycol was slowly added to the filtrate, and the temperature was controlled at 30 ° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 19 was 88.1%, and the yield was 58%. The purity of the tetracyclic aromatic hydrocarbons obtained was 88.6%, and the yield was 59%.

### Example 20

1 g of phenanthrene, 1 g of anthracene, 0.5 g of pyrene and 0.5 g of chrysene were mixed in dichloromethane, stirred and heated at room temperature. The dichloromethane was distilled to remove the dichloromethane to obtain a mixture of phenanthrene, anthracene, pyrene and chrysene. An imidazole-based mixed solvent (wherein the imidazole-based mixed solvent was a mixture of ethylimidazole and ethylene glycol, and the concentration of ethylimidazole was 70 mol %) and a mixture of phenanthrene, anthracene, pyrene and chrysene were mixed in a mass ratio of 3:1, and then stirred at 40° C. Finally, the mixture was filtered to obtain a filter cake (tricyclic aromatic hydrocarbons) and a filtrate.

Ethylene glycol was slowly added to the filtrate, and the temperature was controlled at 30 ° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 20 was 89.1%, and the yield was 60%. The purity of the tetracyclic aromatic hydrocarbons obtained was 92.5%, and the yield was 61%.

### Example 21

1 g of phenanthrene, 1 g of anthracene, 0.2 g of fluorene, 0.5 g of pyrene and 0.5 g of chrysene were mixed in dichloromethane, stirred and heated at room temperature. The dichloromethane was distilled to remove the dichloromethane to obtain a mixture of phenanthrene, anthracene, fluorene, pyrene and chrysene. An imidazole-based mixed solvent (wherein the imidazole-based mixed solvent was a mixture of ethylimidazole and heptanediol, and the concentration of ethylimidazole was 80 mol %) and a mixture of phenanthrene, anthracene, fluorene, pyrene and chrysene were mixed in a mass ratio of 6:1, and then stirred at 50° C. Finally, the mixture was filtered to obtain a filter cake (tricyclic aromatic hydrocarbons) and a filtrate.

Ethylene glycol was slowly added to the filtrate, and the temperature was controlled at 30° C. during the process. The addition was stopped until the solid mass becomes constant. Finally, the imidazole-based mixed solvent and the tetracyclic aromatic hydrocarbon compound were obtained by washing and filtering. The purity of the tricyclic aromatic hydrocarbons obtained in Example 21 was 87.1%, and the yield was 57%. The purity of the tetracyclic aromatic hydrocarbons obtained was 90.5%, and the yield was 60%.

According to the foregoing embodiments, the present invention provides a separation method for polycyclic aromatic hydrocarbons, wherein a mixed solvent and a to-be-separated mixture are first mixed and then filtered to obtain a filter cake and a filtrate, and then a back extraction agent and the filtrate are mixed and separated, wherein the mixed solvent is a naphthalene-based mixed solvent or an imidazole-based mixed solvent, and the to-be-separated mixture is a mixture containing bicyclic aromatic hydrocarbons and tricyclic aromatic hydrocarbons or a mixture containing tricyclic aromatic hydrocarbons and tetracyclic aromatic hydrocarbons. According to the present invention, a mixture containing bicyclic aromatic hydrocarbons and tricyclic aromatic hydrocarbons is separated by using a naphthalene-based mixed solvent, and a mixture containing tricyclic aromatic hydrocarbons and tetracyclic aromatic hydrocarbons is separated by using an imidazole-based mixed solvent, and the naphthalene-based mixed solvent or the imidazole-based mixed solvent can be recycled. In addition, the separation method provided by the present invention has simple steps, low energy consumption and a simple process flow.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A separation method for polycyclic aromatic hydrocarbons, comprising the following steps:
1) mixing a mixed solvent and a to-be-separated mixture, and filtering to obtain a filter cake and a filtrate; and
2) mixing a back extraction agent and the filtrate and then performing a separation; wherein
the mixed solvent is a naphthalene-based mixed solvent or an imidazole-based mixed solvent; and
the to-be-separated mixture is a mixture containing bicyclic aromatic hydrocarbons and tricyclic aromatic hydrocarbons or a mixture containing the tricyclic aromatic hydrocarbons and tetracyclic aromatic hydrocarbons.

2. The separation method according to claim 1, wherein in the step 1), a mass ratio of the mixed solvent to the to-be-separated mixture is (1-8):1.

3. The separation method according to claim 1, wherein the mixture containing the bicyclic aromatic hydrocarbons and the tricyclic aromatic hydrocarbons is separated by using the naphthalene-based mixed solvent, wherein the naphthalene-based mixed solvent is formed by mixing an alcohol compound and a naphthyl compound.

4. The separation method according to claim 3, wherein a concentration of the naphthyl compound is 2-20 mol %.

5. The separation method according to claim 3, wherein the naphthyl compound comprises one or more of naphthylacetonitrile, naphthylacetic acid, naphthylacetamide, naphthylmethanol, naphthylethanol, naphthylamine, and naphthol.

6. The separation method according to claim 1, wherein the mixture containing the tricyclic aromatic hydrocarbons and the tetracyclic aromatic hydrocarbons is separated by using the imidazole-based mixed solvent, wherein the imidazole-based mixed solvent is formed by mixing an alcohol compound and an imidazole compound.

7. The separation method according to claim 6, wherein the alcohol compound comprises one or more of ethylene glycol, propylene glycol, pentanediol, heptanediol, and octanediol.

8. The separation method according to claim 6, wherein the imidazole compound comprises one or more of methylimidazole, ethylimidazole, propylimidazole, and butylimidazole.

9. The separation method according to claim 7, wherein a concentration of the imidazole compound is 10-80 mol %.

10. The separation method according to claim 2, wherein the mixture containing the bicyclic aromatic hydrocarbons and the tricyclic aromatic hydrocarbons is separated by using the naphthalene-based mixed solvent, wherein the naphthalene-based mixed solvent is formed by mixing an alcohol compound and a naphthyl compound.

11. The separation method according to claim 10, wherein a concentration of the naphthyl compound is 2-20 mol %.

12. The separation method according to claim 10, wherein the naphthyl compound comprises one or more of naphthylacetonitrile, naphthylacetic acid, naphthylacetamide, naphthylmethanol, naphthylethanol, naphthylamine, and naphthol.

13. The separation method according to claim 2, wherein the mixture containing the tricyclic aromatic hydrocarbons and the tetracyclic aromatic hydrocarbons is separated by using the imidazole-based mixed solvent, wherein the imidazole-based mixed solvent is formed by mixing an alcohol compound and an imidazole compound.

14. The separation method according to claim 13, wherein the alcohol compound comprises one or more of ethylene glycol, propylene glycol, pentanediol, heptanediol, and octanediol.

15. The separation method according to claim 13, wherein the imidazole compound comprises one or more of methylimidazole, ethylimidazole, propylimidazole, and butylimidazole.

16. The separation method according to claim 14, wherein a concentration of the imidazole compound is 10-80 mol %.

17. The separation method according to claim 8, wherein a concentration of the imidazole compound is 10-80 mol %.

18. The separation method according to claim 15, wherein a concentration of the imidazole compound is 10-80 mol %.
